(19) United States
(12) Patent Application Publication
(10) Pub. No.: US 2022/0195140 A1
(43) Pub. Date: Jun. 23, 2022

(54) CONVERSION OF PLASTICS TO MONOMERS BY ACIDIC CATALYTIC PYROLYSIS

(71) Applicant: UOP LLC, Des Plaines, IL (US)

(72) Inventors: Yili Shi, Buffalo Grove, IL (US); Hayim Abrevaya, Kenilworth, IL (US); Scott Lyle Nauert, Chicago, IL (US); David Gray, Homer Glen, IL (US)

(21) Appl. No.: 17/546,898

(22) Filed: Dec. 9, 2021

Related U.S. Application Data
(60) Provisional application No. 63/129,850, filed on Dec. 23, 2020.

(51) Int. Cl. C08J 11/16; C07C 4/22

(52) U.S. Cl. CPC C08J 11/16; C07C 2521/04; C08J 2323/06; C07C 4/22

(57) ABSTRACT

A plastic catalytic pyrolysis process that can produce high yields of ethylene, propylene and other light olefins from waste plastics is disclosed. The plastic feed is catalytically pyrolyzed at high silica-to-alumina ratios and elevated temperature to produce high ratios of gas to liquid which results in high light olefin monomer selectivity. The catalytic pyrolysis process can be operated in a single stage or a two-stage process.

### CROSS-REFERENCE TO RELATED APPLICATIONS

This application claims priority from U.S. Provisional Application No. 63/129,850, filed Dec. 23, 2020, which is incorporated herein in its entirety.

### FIELD

The field is the recycling of plastic materials to produce monomers.

### BACKGROUND

The recovery and recycle of waste plastics is held with deep interest by the general public which has been participating in the front end of the process for decades. Past plastic recycling paradigms have involved mechanical recycling. Mechanical recycling entails sorting, washing and melting recyclable plastic articles to molten plastic materials to be remolded into a new clean article. However, this mechanical recycling process has not proven economical. The melt and remolding paradigm has encountered several limitations, including economic and qualitative. Collection of recyclable plastic articles at materials recovery facilities inevitably includes non-plastic articles that had to be separated from the recyclable plastic articles. Similarly, collected articles of different plastics have to be separated from each other before undergoing melting because the articles molded of different plastics would not typically have the quality of an article molded of the same plastic. Separation of collected plastic articles from non-plastic articles and then into the same plastics added expense to the process that made it less economical. Additionally, recyclable plastic articles have to be properly cleaned to remove non-plastic residues before melting and remolding which also added to the expense of the process. The recovered plastic also does not possess the quality of virgin grade resins. The burdensome economics of the plastic recycling process and the lower quality of recycled plastic have prevented widespread renewal of this renewable resource.

A paradigm shift has enabled the chemical industry to rapidly respond with new chemical recycling processes for recycling waste plastics. The new paradigm is to chemically convert the recyclable plastics in a pyrolysis process operated at about 350 to 600° C. to liquids. The liquids can be refined in a refinery to fuels, petrochemicals and even monomers that can be re-polymerized to make virgin plastic resins. The pyrolysis process still requires separation of collected non-plastic materials from plastic materials fed to the process, but cleaning and perhaps sorting of plastic materials may not be as critical in chemical recycling.

Higher temperature pyrolysis is under investigation and is viewed as a route to convert plastics directly to monomers without further refining. Conversion of plastics back to monomers presents a circular way of recycling a renewable resource that as of yet has not been fully economically developed.

Catalytic pyrolysis of plastics is in exploration. In a single stage catalytic pyrolysis process, the plastic feed and the catalyst are heated together to catalytic reaction temperature. In a two-stage catalytic pyrolysis process, the plastic feed is heated to pyrolysis temperature to produce a vaporized pyrolysis stream which is then contacted with to the catalyst. These processes have achieved only lower yields of monomers, instead focusing on liquid yields. What is needed is a viable catalytic process to convert plastic articles back to monomers.

### BRIEF SUMMARY

This disclosure describes a plastic pyrolysis process that can produce high yields of monomers from waste plastics. In a two-stage process, a plastic feed is pyrolyzed at an elevated temperature to produce a vaporized pyrolysis stream which is then contacted with a catalyst having a silica-to-alumina ratio of at least 80 to produce a catalytic product stream comprising monomers. The elevated temperature may be at least 450° C. In an alternative high-temperature two-stage process, the plastic feed is pyrolyzed at a temperature of at least 600° C. before the contacting the vaporized pyrolysis gas stream with a catalyst having a silica-to-alumina ratio of above 50. In an alternative single-stage process, the catalyst and plastic feed can be contacted with a catalyst having a silica-to-alumina ratio of at least 40 at a reaction temperature of at least 500° C.

### BRIEF DESCRIPTION OF THE DRAWINGS

FIG. 1 is a schematic drawing of a process of one embodiment of the present disclosure.

FIG. 2 is a schematic drawing and graph illustrating an example of the present disclosure.

### DEFINITIONS

The term “communication” means that fluid flow is operatively permitted between enumerated components, which may be characterized as “fluid communication”.

The term “downstream communication” means that at least a portion of fluid flowing to the subject in downstream communication may operatively flow from the object with which it fluidly communicates.

The term “upstream communication” means that at least a portion of the fluid flowing from the subject in upstream communication may operatively flow to the object with which it fluidly communicates.

The term “direct communication” means that fluid flow from the upstream component enters the downstream component without passing through any other intervening vessel.

The term “indirect communication” means that fluid flow from the upstream component enters the downstream component after passing through an intervening vessel.

The term “bypass” means that the object is out of downstream communication with a bypassing subject at least to the extent of bypassing.

The term “predominant”, “predominance” or “predominate” means greater than 50%, suitably greater than 75% and preferably greater than 90%.

The term “carbon-to-gas mole ratio” means the ratio of mole rate of carbon atoms in the plastic feed stream to the mole rate of gas in the diluent gas stream. For a batch process, the carbon-to-gas mole ratio is the ratio of moles of carbon atoms in the plastic in the reactor to the moles of gas added to the reactor.

The term “silica-to-alumina ratio” means the mole ratio of SiO₂ to Al₂O₃ present in a sub stance.

### DETAILED DESCRIPTION

We have discovered a process for converting plastics to monomers by integrating a plastics catalytic pyrolysis process at elevated temperature to produce light olefinic monomers. The plastic feed can comprise polyolefins such as polyethylene and polypropylene. Any type of polyolefin plastic is acceptable even if mixed with other monomers randomly or as a block copolymer. Other polymers that can be used with or without other polymers include polyethylene terephthalate, polyvinyl chloride, polystyrene, polyamides, acrylonitrile butadiene styrene, polyurethane and polysulfone. Many different plastics can be used in the feed because the process pyrolyzes the plastic feed to smaller molecules including light olefins.

In an embodiment, the plastic feed stream may be obtained from a materials recycling facility (MRF) that is otherwise sent to a landfill. The plastic feed may be compressed plastic articles from a separated bail of compacted plastic articles. The plastic articles can be chopped into plastic chips or particles.

An augur or an elevated hopper may be used transport the plastic feed as whole articles, chips or particles into a pyrolysis reactor. Plastic articles, chips or particles may be heated to above the plastic melting point into a melt and injected or augured into the reactor. An augur may operate in such a way as to move whole plastic articles into the reactor and simultaneously melt the plastic articles in the augur by friction or by indirect heat exchange into a melt which enters the reactor in a molten state. Chips or particles may be melted during auguring to the reactor 12 or they may be kept below melting temperature and augured into the reactor as a solid.

The plastic feed may be processed in a single-stage or a two-stage process. In a single-stage process, plastic feed is pyrolyzed and catalyzed simultaneously. In a two-stage process, the plastic feed is pyrolyzed and the resulting vaporized pyrolysis stream is catalyzed.

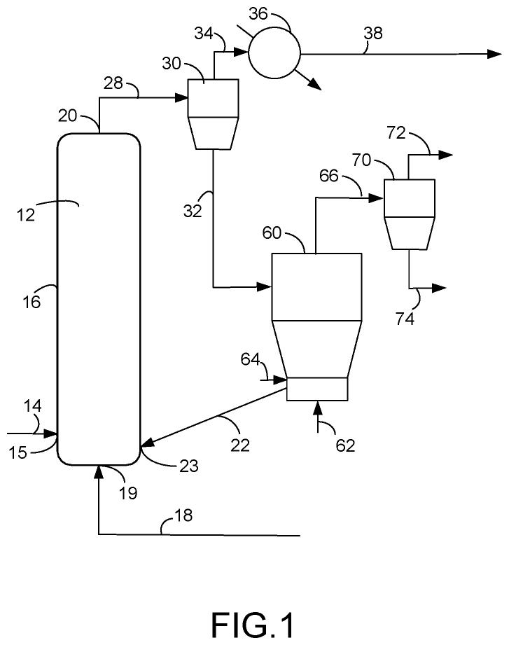

In an embodiment, a single-stage catalytic pyrolysis process may be conducted in fluidized reactor 12 as shown in FIG. 1. The plastic feed may be injected into the reactor 12. In the reactor 12, the plastic feed may be contacted with a diluent gas stream. The diluent gas stream is preferably inert, such as nitrogen, but it may be a hydrocarbon gas. Steam is a preferred diluent gas stream. The diluent gas stream separates reactive olefin products from each other to preserve the selectivity to light olefins thus avoiding oligomerization of light olefins to higher olefins or over cracking to light gas.

The diluent gas stream may be provided through a distributor from a diluent line 18 and may be distributed through a diluent inlet 19. The diluent gas stream may be blown into the reactor 12 through the diluent inlet 19. The diluent inlet 19 may be in a bottom of the reactor 12. The diluent gas stream may be used to impel plastic feed from the feed inlet 15 of the reactor 12 to an outlet 20 of the reactor. In an aspect, the feed inlet 15 may be at a lower end of the reactor 12, and the outlet 20 may be at an upper end of the reactor. The interior of the wall 16 of the reactor 12 may be coated with refractory lining to insulate the reactor and conserve its heat.

In the single stage process, the catalytic pyrolysis reaction temperature should be at least 500° C., suitably at least 525° C. and preferably at least 530° C. To achieve this reaction temperature the plastic feed may be heated to a catalytic pyrolysis temperature of about 500° C. to about 700° C., suitably at least about 525° C. and preferably at least about 530° C. The catalytic pyrolysis temperature will be much higher than the melting temperature of the plastic at which the plastic may be fed to the reactor 12. In a single-stage process, plastic feed is preferably heated to catalytic pyrolysis temperature after entering the reactor 12. In an embodiment, the plastic feed is heated to catalytic pyrolysis temperature by contacting it with a stream of hot catalyst particles. The stream of catalyst may be fed to the reactor in a catalyst line 22 through a catalyst inlet 23. In an aspect, the catalyst inlet 23 may be located between the diluent inlet 19 and the plastic feed inlet 15. The lift gas stream will then contact and move the stream of hot catalyst into contact with the plastic feed from feed line 14 through feed inlet 15.

It is also contemplated that some or all of the diluent gas stream may impel the catalyst into the reactor in which case the diluent gas stream and the stream of catalyst may enter the reactor 12 through the same inlet. Additionally, the diluent gas stream may impel the plastic feed into the reactor 12 in which case the diluent gas stream and the plastic feed stream may enter the reactor through the same inlet. It is also contemplated that the plastic feed stream and the stream of catalyst may be impelled into the reactor 12 by some or all of the diluent gas stream, in which case at least some of the diluent stream, the plastic feed stream and the stream of catalyst may all enter the reactor 12 through the same inlet.

It another embodiment, the feed inlet 15 and the catalyst inlet 23 may be located in an upper end of the reactor from which they can fall together in a downer reactor arrangement (not shown). The diluent gas stream would not function in this embodiment to upwardly fluidize the feed and catalyst.

Upon heating the plastic feed to catalytic pyrolysis temperature, the plastic feed vaporizes and catalytically pyrolyzes to smaller molecules including light olefins. Diluent gas from the diluent inlet 19 may be used to impel the catalyst from the catalyst inlet 23 up into contact with the plastic feed stream from the feed inlet 15. The vaporization and conversion to a greater number of moles both increase volume causing rapid movement of feed and pyrolysis product toward the reactor outlet 20. Due to the volume expansion of the plastic feed, a diluent gas stream is not necessary to rapidly move feed and product to the outlet. However, diluent gas also serves to separate product olefins from each other and from catalyst particles to prevent oligomerization and over-cracking which both diminish light olefin selectivity. So, the diluent gas stream may be employed to move the plastic feed stream while undergoing pyrolysis while in contact with the stream of hot catalyst toward the reactor outlet 20. In an aspect, we have found that the diluent gas stream can be introduced at a high carbon-to-gas mole ratio of about 0.6 to about 20. The carbon-to-gas mole ratio may be at least about 0.7, suitably at least about 0.8, more suitably at least about 0.9 and most suitably at least about 1.0. In an aspect, the carbon-to-gas mole ratio may not exceed about 15, suitably may not exceed about 12, more suitably may not exceed about 9 and most suitably may not exceed about 7 and preferably will not exceed about 5. The high carbon-to-gas mole ratio importantly reduces the amount of diluent gas that must be separated from other gases including product gases.

Spherical particles may be most easily lifted or fluidized by the diluent gas stream from the inlet 19. So, the catalyst may be carried on spherical alpha alumina particles. The spherical alpha alumina may be formed by spray drying an alumina and catalyst solution, followed by calcining it at a temperature that converts the alumina to the α-alumina crystalline phase. In an embodiment, the catalyst particles should have a smaller average diameter than the plastic articles, chips, particles or melt fed to the reactor. The average diameter of the catalyst particles refers to the largest average diameter of the catalyst particles. The plastic melt may enter the reactor in molten globs that will typically have a larger average diameter than the catalyst particles.

The plastic feed may be catalytically pyrolyzed by rapidly imparting a relatively high temperature to feedstocks for a very short residence time, typically about 0.5 seconds to about 0.5 minutes, and then rapidly reducing the temperature of the pyrolysis products before chemical equilibrium can occur. By this approach, the structures of the polymers are broken into reactive chemical fragments that are initially formed by depolymerization and volatilization reactions, but do not persist for any significant length of time. Catalytic pyrolysis can be carried out in a variety of pyrolysis reactors such as fluidized bed pyrolysis reactors and circulating fluidized bed reactors.

The pyrolysis process produces a carbon-containing solid called char, coke that accumulates on the catalyst particles and pyrolysis gases comprising hydrocarbons including olefins and hydrogen gas.

The catalyst particles and the plastic feed stream may be fluidized in the reactor by the diluent gas stream. The plastic feed stream and the stream of catalyst may be fluidized by the diluent gas stream continually entering the reactor 12 through the diluent inlet 19. The catalyst and plastic feed stream can be fluidized in a dense bubbling bed. The molten plastic and catalyst may congeal together into globs until the plastic in the glob fully pyrolyzes to gas. In a bubbling bed, the diluent gas stream and vaporized plastic form bubbles that ascend through a discernible top surface of a dense particulate bed. Only catalyst entrained in the gas exits the reactor with the vapor. For a plastic feed that is fluidized and fed to the reactor 12, the superficial velocity of the gas in a bubbling bed is typically less than 3.4 m/s (11.2 ft/s) and the density of the dense bed is typically greater than 475 kg/m³ (49.6 lb/ft³). For a solid plastic feed that is fed as solid particles or fed as a melt to the reactor 12, such that the plastic feed and catalyst congeal into globs, the superficial velocity for solid plastic feed will be less than 2.7 m/s (9 ft/s) and the density of the bed will be greater than 274 kg/m³ (17.1 lb/ft). The mixture of catalyst and gas is heterogeneous with pervasive vapor bypassing of catalyst. In the dense bubbling bed, gases will exit the reactor outlet 20; whereas, the solid catalyst and char may exit from a bottom outlet (not shown) of the reactor 12.

In an aspect, the reactor 12 may operate in a fast-fluidized flow regime or in a transport or pneumatic conveyance flow regime with a dilute phase of catalyst particles. In a further aspect, the reactor 12 may operate as a riser reactor. In a fast-fluidized flow and transport flow regime, the stream of globs of catalyst particles and molten plastic undergoing pyrolysis and gaseous pyrolyzed plastic and the diluent gas stream will flow upwardly together. In both cases, a quasi-dense bed of plastic and catalyst particle globs will undergo pyrolysis at the bottom of the reactor 12. The globs of plastic and catalyst will transport upwardly upon sufficient size reduction due to pyrolysis. The diluent gas stream may lift the plastic feed stream and the stream of catalyst. The mixture of gases and the catalyst may be discharged together from the reactor outlet 20 if a separator 30 is located outside of the reactor 12. If a separator 30 is located in the reactor 12, the gases will be discharged from the reactor outlet 20 and the catalyst and char will exit from an additional catalyst outlet. Typically, the reactor outlet 20 which discharges the catalyst will be above the catalyst inlet 23. Furthermore, separation of the catalyst from the gaseous products will be conducted above the catalyst inlet 23 and/or the feed inlet 15 in transport and fast-fluidized flow regimes.

The density for a fluid feed in the fast-fluidized flow regime will be between at least about 274 kg/m³ (17.1 lb/ft³) to about 475 kg/m³ (49.6 lb/ft³) and in a transport flow regime will be no more than 274 kg/m3 (17.1 lb/ft³). The density for a plastic feed that congeals into globs in the fast-fluidized flow regime will be between about 120 kg/m³ (7.5 lb/ft³) and 274 kg/m³ (17.1 lb/ft³) and in a transport flow regime will be no more than 120 kg/m³ (7.5 lb/ft³). The superficial gas velocity will typically be about 2.7 m/s (9 ft/s) to about 8.8 m/s (28.9 ft/s) in a fast-fluidized flow regime for globs of catalyst congealed with plastic. In a transport flow regime, the superficial gas velocity will be at least about 8.8 m/s (28.9 ft/s) for globs of catalyst congealed with plastic. The superficial gas velocity will typically be about 3.4 m/s (11.2 ft/s) to about 7.3 m/s (15.8 ft/s) in a fast-fluidized flow regime for fluidized plastic feed. In a transport flow regime, the superficial gas velocity will be at least about 7.3 m/s (15.8 ft/s) for fluidized plastic feed. The diluent gas stream and product gas ascend in a fast-fluidized flow regime but the hot catalyst may slip relative to the gas and the gas can take indirect upward trajectories. In a transport flow regime, less of the solids will slip. In some fluidized reactors, such as in a riser reactor, residence time for the plastics and product gas in the reactor may be about 1 to about 20 seconds and typically no more than about 10 seconds.

The reactor effluent comprising catalyst, diluent gas stream and pyrolyzed product gas may exit the reactor 12 through the reactor outlet 20 in a reactor effluent line 28 and be transported to a separator 30. In an aspect, the separator 30 may be located in the reactor 12. If the separator 30 is located in the reactor 12, the catalyst, the diluent gas stream and the pyrolyzed product gas will enter into the separator 30. The reactor effluent in line 28 will be at a temperature of about 450° C. to about 700° C. and a pressure of about 1.5 to 2.0 bar (gauge).

The separator 30 may be a cyclonic separator that utilizes centripetal acceleration to separate the catalyst from pyrolyzed gaseous products. The reactor effluent line 28 may tangentially cast reactor effluent into the cyclone separator 30 in a typically horizontally angular trajectory causing the reactor effluent to centripetally accelerate. The centripetal acceleration causes the denser catalyst to gravitate outwardly. The catalyst particles lose angular momentum and descend in the cyclone separator 30 into a lower catalyst bed and exit through a heat carrier dip line 32. The less dense gaseous product ascends in the cyclone 30 and are discharged through transfer line 34. In an aspect, pyrolysis gas products may be stripped from catalyst in line 32 by adding a stripping gas to a lower end of the dip line 32. In this embodiment, stripping gas and stripped pyrolysis gases would exit the separator 30 in the transfer line 34.

In an embodiment, a catalytic pyrolysis product stream in the transfer line 34 may be immediately quenched to prevent and terminate hydrogen transfer reactions and over-cracking which may occur to diminish light olefin monomer selectivity in the high-temperature pyrolysis product stream. Quenching should occur as soon as possible after separation of the pyrolysis gas product from the catalyst. Quenching should occur within 1 second of separation of pyrolysis gas from catalyst and preferably within 1 second of exit from the reactor 12.

Quenching may be effected in the following manner although other quenching processes are contemplated. The catalytic pyrolysis product stream may be quench cooled by indirect heat exchange perhaps with water to make steam for the diluent gas stream in a transfer line exchanger 36. The quenched catalytic pyrolysis product stream in line 38 may be at a temperature of about 400° C. to about 500° C. In an aspect, the quenched catalytic pyrolysis product stream may be completely quenched by indirect heat exchange with water to produce steam in the transfer line exchanger 36. If the quenched catalytic pyrolysis product stream is completely quenched by indirect heat exchange, the completely cooled catalytic pyrolysis product stream may exit the transfer line exchanger 36 at about 30° C. to about 60° C. and around atmospheric pressure, 1 to about 1.3 bar (gauge), so lighter components of the vaporous high-temperature pyrolysis product stream can condense.

Turning back to the separator 30, the catalyst stream in the dip line 32 may have accumulated coke from the catalytic pyrolysis process. Moreover, char residue from the catalytic pyrolysis process may also end up with the catalyst in the line 40. The catalyst particles have also given off much of their heat in the reactor 12 and need to be reheated. Therefore, the dip line 32 delivers the catalyst stream with char to a regenerator 60.

In aspect, a predominance of catalyst entering the regenerator 60 passes through the separator 30. In an embodiment, all of the catalyst entering the regenerator 60 passes through the separator 30.

The catalyst and char are fed to the regenerator 60 and contacted with an oxygen supply gas in line 62 such as air to combust char and the coke on the cooler catalyst. The regenerator 60 is a separate vessel from the reactor 12. The coke is burned off the spent catalyst by contact with the oxygen supply gas at combustion conditions in the regenerator 60. Heat of combustion serves to reheat the catalyst. About 10 to about 15 kg of air are required per kg of coke burned off of the catalyst. A fuel gas stream in line 64 may also be added to the regenerator 60 if necessary, to produce sufficient heat to drive the pyrolysis reaction in the reactor 12. The fuel gas may be obtained from paraffins recovered from the catalytic product stream in line 38. Exemplary regeneration conditions include a temperature from about 700° C. to about 1000° C. and a pressure of about 1 to about 5 bar (absolute) in the regenerator 60.

A stream of regenerated catalyst is recycled from the regenerator 60 to the reactor 12 in line 22 through the catalyst inlet 23 at a temperature of the regenerator 60. Flue gas and entrained char exit the regenerator in line 66 and are delivered to a cyclone 70 which separates exhaust gas in an overhead line 72 from a solid ash product in line 74.

In the two-stage catalytic pyrolysis process, plastic feed is pyrolyzed and the vaporized pyrolysis stream is subjected to catalytic pyrolysis. In the first step of the two-stage process, the plastic feed is subjected to pyrolysis at elevated temperature. The pyrolysis reactor may be a continuous stirred tank reactor (CSTR), a rotary kiln, an augured reactor or a fluidized bed. In the reactor the plastic feed stream is heated to a temperature that pyrolyzes the plastic feed stream to a pyrolysis product stream. The reactor provides enough residence time for all of the plastic in the plastic feed stream to convert to a vaporized pyrolysis stream.

The pyrolysis reactor may operate at a temperature from about 450° C. (813° F.) to about 700° C. (1292° F.), or preferably about 530° C. (986° F.) to about 600° C. (1112° F.), a pressure from about 0.069 MPa (gauge) (10 psig) to about 1.38 MPa (gauge) (200 psig), or preferably about 0.138 MPa (gauge) (20 psig) to about 0.55 MPa (gauge) (80 psig). For example, a heated inert, diluent gas stream may be flowed through or over the plastic feed to heat the plastic feed to pyrolysis temperature. Alternatively, the plastic feed and diluent gas may be heated together and the pyrolysis gas driven off the plastic feed may be carried in the diluent gas stream. An inert diluent gas may comprise nitrogen or steam. The diluent gas stream may also be used to fluidize the plastic feed stream. The diluent gas stream may be added to the reactor at a rate of about 17 Nm³/m³ (100 scf/bbl) to about 850 Nm³/m³ plastic feed (5,000 scf/bbl), or more preferably about 170 Nm³/m³ (1000 scf/bbl) to about 340 Nm³/m³ plastic feed (2000 scf/bbl). The diluent gas stream may serve to reduce impure gas partial pressure in the vaporized pyrolysis gas stream.

The pyrolysis reactor may contain a guard bed to trap solids or adsorb impurities in the pyrolysis stream. An example of an adsorbent for the guard bed is alumina. The pyrolysis reaction converts the plastic feed to an intermediate molecular composite which can be more readily catalyzed in the catalytic reaction step. A vaporized pyrolysis stream may be withdrawn from the pyrolysis reactor. In an embodiment, the vaporized pyrolysis stream is carried in the inert gas stream to the second catalyst stage of the process.

The vaporous pyrolysis stream may be transported to a catalytic reactor. In an embodiment, the catalytic reactor may be a second catalyst bed in a vessel downstream of a guard bed or pyrolytic reactor section. In another embodiment, the vaporized pyrolysis stream in the diluent gas stream or by itself may be sprayed into a bed of catalyst in the catalytic reactor to fluidize the catalyst bed. Alternatively, another gas stream, such as a diluent gas stream, may be sprayed into the catalyst bed to fluidize the catalyst and the vaporized pyrolysis stream may be distributed into the fluidized catalyst. The catalytic reactor may be operated according to the reactor 16 of FIG. 1.

In the two-stage process, the temperature in the catalytic reactor may be higher than in the pyrolysis reactor. The catalytic reactor may be at a higher temperature than in the pyrolysis reactor because in the pyrolysis reactor, the plastic feed melts, vaporizes and partially cracks which has an endothermic effect and absorbs much heat from the heater. The vaporized pyrolysis stream entering the catalytic reactor may then predominantly undergo catalytic reactions of which some are endothermic but absent the melting and vaporization already achieved in the pyrolysis reactor, the catalytic reactor demands less heat from the heater and thus rises to a higher reaction temperature.

Similar catalyst can be used in both single and two-stage processes. In an embodiment, the catalyst is acidic. The catalyst may be a molecular sieve. In an embodiment, the catalyst may be an acidic molecular sieve. In a further embodiment, the catalyst is a zeolitic or a non-zeolitic molecular sieve. In an embodiment, the catalyst is a zeotype material. The catalyst may be a zeolite with 10-membered rings such as having an MFI structure. The catalyst may have 10-membered rings but pores smaller than MFI such as TON and MTT structures and Ferrierite. A zeolite with 8-membered rings or 12-membered rings such as Y-zeolite and beta zeolite may also be suitable.

It is important that the catalyst have low acidity. The acidity of the catalyst can be characterized by a silica-to-alumina ratio. A combination of reaction temperature and catalyst acidity may be necessary to obtain sufficient monomer production at lower acidities. The same catalyst may be used in the single-stage catalytic pyrolysis process as used in the two-stage catalytic pyrolysis. We have found in the single-stage process, the silica-to-alumina ratio can be as low as at least 40 and preferably at least above 50 if the catalytic reaction temperature is at least 500° C., suitably at least 525° C. and preferably at least 530° C. In the single-stage process, if the silica-to-alumina ratio of the catalyst is at least about 80, the catalytic reaction temperature may be just above 450° C., suitably at least 475° C. and preferably at least 500° C. In the two-stage process, the silica-to-alumina ratio of the catalyst should be at least about 80 as long as the catalytic reaction temperature is greater than 450° C., typically at least 475° C., suitably at least 500° C. and more suitably at least 525° C. and preferably at least 530° C. The silica-to-alumina ratio of the catalyst should be at least just above 50 when the catalytic reaction temperature is greater than 600° C. and suitably at least 625° C. and preferably at least 630° C.

The catalyst with a high silica-to-alumina ratio provides a lower acidity catalyst due to less alumina concentration in the catalyst. With less alumina concentration the acid sites are not as close together thus minimizing side reactions which can be caused by acid sites being near each other. To compensate for lower acidity, temperature should be elevated or a lower weight space velocity should be employed to achieve sufficient cracking to monomers.

Catalytic reactions occurring in the catalytic reactor include: 1) cracking reactions involving carbon-carbon scission which can produce desired light olefins, 2) aromatization in catalyst pores producing aromatics which may take the form of coke, and 3) hydride transfer recombination reactions which produce paraffins. Reaction 1) should be maximized while reactions 2) and 3) should be minimized. The low acidity catalyst operates to impair reactions 2) and 3) preferentially compared to reaction 1), and the elevated temperature preferentially promotes reaction 1) compared to reactions 2) and 3). We have found that decreasing the silica-to-alumina ratio increases the C₁-C₄ alkane yield at expense of C₂-C₄ olefin yield.

The smaller pore molecular sieves utilizing 8 membered rings may limit reactions 2) and 3). Large pore molecular sieves with 12-membered rings may be effective so long as the silica-to-alumina ratio is at least 80. The crystallite size of the catalyst can range from 2 nm to 6 μm and typically from about 1 to about 3 The weight hourly space velocity should be about 1 hr⁻¹ to about 20 hr⁻¹, and preferably at least 2 hr⁻¹, in the catalytic reactor. The gas residence time in the catalytic reactor should be short to avoid over-cracking, typically about 0.5 seconds to about 0.5 minutes. The catalyst-to-plastic ratio should range from about 5 to about 80 if a fluidized bed reactor is employed.

In an aspect, the catalyst bed comprises a single catalyst type rather than a mixture of catalyst to provide a uniform chemistry as much as possible. The catalyst in the reactor should comprise at least 70 wt %, suitably at least 75 wt %, more suitably at least 80 wt %, even more suitably at least 85 wt %, preferably at least 90 wt % and most preferably at least 95 wt % of a single catalyst type.

The catalytic pyrolysis process disclosed produces vastly more gas than liquids. The catalytic process results in a gas fraction of at least about 75% and preferably at least 90% and a gas-to-liquid ratio of at least 15 and preferably about 16 to about 300. The gas fraction is the percentage of gas relative to the total product including gas, liquid and coke solids. The gas-to-liquid ratio excludes consideration of coke.

### Example

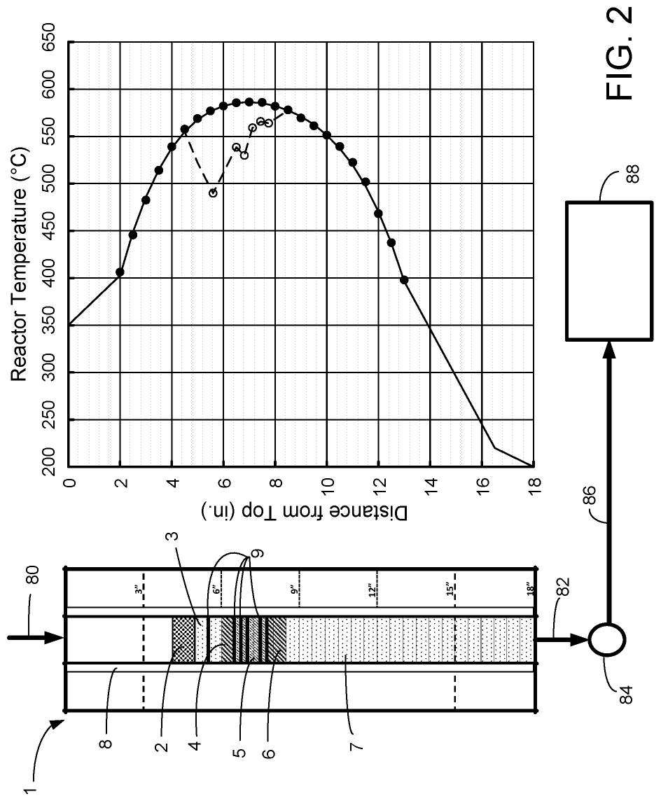

We conducted a pyrolysis reaction of high-density polyethylene (HDPE) plastic feed at elevated temperatures in a two-stage catalytic pyrolysis process. The set-up of a reactor 1 is depicted in FIG. 2. Plastic pellets 2 were stacked onto a top of a guard bed 3 of alumina beads at the top of reactor 1. A bed 4 of quartz separated the guard bed 3 from a top of a catalyst bed 5. Another bed 6 of quartz separates a bottom of the catalyst bed 5 from a bottom bed 7 of alumina beads. The reactor 1 was heated by an external furnace 8 surrounding the reactor. A diluent stream 80 of nitrogen was fed to the top of the reactor 1. The diluent stream picked up pyrolysis gas generated from the plastic pellets and carried it through the reactor 1. The feed rate targeted 75 wt % HDPE pellets and 25 wt % nitrogen gas.

Thermocouples 9 were spaced along the height of the reactor 1 which registered temperatures shown in the graph at the right in FIG. 2 at corresponding heights from the top of the reactor. Unfilled circles show the temperatures registered by the thermocouples 9 at variance from the temperature profile provided by the external furnace 8 due to the net endothermic reactions in the reactor 1. Reactor effluent in line 82 exits the reactor 1 and enters a knock-out pot 84 and is cooled down to 190° C. Uncondensed product gas is fed in line 86 to a gas chromatograph 88 to determine the composition of the reactor effluent.

The catalytic pyrolysis conditions and rough product composition for four runs are shown in Table 1.

TABLE 1

| Run No. | 1120 | 1121 | 1122 | 1123 |
|---|---|---|---|---|
| Catalyst | alpha-Al₂O₃ | MFI | MFI | MFI |
| Silica/Alumina ratio | 0 | 350 | 80 | 40 |
| Cat. loading (g) | — | 4 | 0.5 | 0.6 |
| Furnace Temp. (° C.) | 600 | 600 | 600 | 600 |
| Rx Temp. (° C.) | 584 | 536 | 538 | 575 |
| WHSV (h⁻¹) | — | 4.9 | 44.2 | 28.6 |
| HDPE (g/h) | 17.5 | 19.5 | 22.2 | 17.1 |
| N₂ (g/h) | 7.51 | 7.51 | 7.5 | 7.5 |
| Gas (avg. g/h) * | 7.2 | 14.1 | 17.8 | 10.9 |
| Liquid (avg. g/h) | 3.1 | 0.085 | 0.17 | 0.04 |
| Coke (avg. g/h) | 2.8 | 0.87 | 0.52 | 0.54 |
| Gas Fraction (%) | 54 | 93 | 96 | 95 |
| Gas-Liquid Ratio | 2.3 | 16.5 | 105 | 273 |
| Mass Bal. (avg. %) | 75 | 77 | 83 | 67 |

A gas leak and condensation after the gas chromatograph lowered the gas mass balance. However, gas production is much greater than liquid production by one or two orders of magnitude. The product selectivity from the experiments is shown in Table 2 taken at different time periods which did not produce large differences in selectivity.

TABLE 2

| | | | | | | | | | Alkanes (wt %) | | | |
|---|---|---|---|---|---|---|---|---|---|---|---|---|
| | Time | | | Olefins (wt %) | | | | BTX | C₁- | C₅- | | Coke |
| Run | (hr) | Catalyst | Si/Al₂ | C₂= | C₃= | C₄= | Total | (wt %) | C₄ | C₉ | C₁₀+ | (wt %) |
| 1120 | 0.25 | Al₂O₃ | 0 | 9.6 | 7.3 | 9.3 | 26.2 | 2.4 | 9.4 | 26.9 | 14.5 | 20.5 |
| 1120 | 0.75 | Al₂O₃ | 0 | 9 | 8.1 | 9.5 | 26.6 | 2.3 | 8.7 | 28.1 | 14.2 | 20 |
| 1120 | 1.25 | Al₂O₃ | 0 | 9.2 | 7.7 | 8.5 | 25.4 | 2.2 | 8.6 | 27.5 | 15 | 21.2 |
| 1120 | 1.75 | Al₂O₃ | 0 | 8.9 | 7.4 | 8.6 | 24.9 | 2 | 8.7 | 25.6 | 16 | 22.7 |
| 1120 | 2.25 | Al₂O₃ | 0 | 9.8 | 8.4 | 9.1 | 27.3 | 1.4 | 9.8 | 23 | 16 | 22.5 |
| 1121 | 0.25 | MFI | 350 | 14.9 | 33.1 | 15.3 | 63.3 | 6.3 | 10.8 | 11.2 | 0.1 | 8.26 |
| 1121 | 0.75 | MFI | 350 | 12.5 | 33.3 | 21.4 | 67.2 | 5 | 8.6 | 12.7 | 0.1 | 6.53 |
| 1121 | 1.25 | MFI | 350 | 11.3 | 33.9 | 18.9 | 64.1 | 5.4 | 8.1 | 17.1 | 0.1 | 5.28 |
| 1121 | 1.75 | MFI | 350 | 10.2 | 32.7 | 24.1 | 67 | 4.6 | 7.1 | 15.7 | 0.1 | 5.4 |
| 1121 | 2.25 | MFI | 350 | 11.5 | 34 | 18.8 | 64.3 | 4.2 | 8.2 | 17.2 | 0.1 | 6 |
| 1122 | 0.25 | MFI | 80 | 11.1 | 25.8 | 19 | 55.9 | 6.1 | 18.9 | 15.6 | 0 | 3.6 |
| 1122 | 0.75 | MFI | 80 | 9.7 | 24.7 | 19.8 | 54.2 | 5.5 | 18.5 | 19.1 | 0 | 2.6 |
| 1122 | 1.25 | MFI | 80 | 11.6 | 27.2 | 19.2 | 58 | 5 | 18 | 15.7 | 0 | 3.2 |
| 1122 | 1.75 | MFI | 80 | 10.2 | 28 | 21.8 | 60 | 5.3 | 15.2 | 16.8 | 0 | 2.7 |
| 1123 | 0.25 | MFI | 40 | 13.9 | 23.2 | 14.1 | 51.2 | 8.6 | 25.5 | 10 | 0 | 4.75 |

Ethylene ranged from about 10 to about 20 wt %, and more precisely, about 11 to about 15 wt % of the product with a silica-to-alumina ration of at least 40. Propylene ranged from about 20 to about 40 wt %, and more precisely, about 23 to about 34 wt % of the product with a silica-to-alumina ration of at least 40. Butenes ranged from about 10 to about 30 wt %, more precisely about 14 to about 24 wt % of the product. Benzene, toluene and xylenes ranged from about 3 to about 10 wt %, and more precisely about 4 to about 9 wt % of the product. The balance of the product comprised alkanes and coke. Decreasing Si/Al₂ ratio increases the C₁-C₄ alkane yield at expense of C₂-C₄ olefin yield.

### SPECIFIC EMBODIMENTS

While the following is described in conjunction with specific embodiments, it will be understood that this description is intended to illustrate and not limit the scope of the preceding description and the appended claims.

A first embodiment of the disclosure is a process for converting plastics to monomers comprising heating a plastic feed to a temperature of about 450 to about 700° C. to pyrolyze the plastic feed to provide a vaporized pyrolysis stream; contacting the vaporized plastic pyrolysis stream with a catalyst having a silica-to-alumina ratio of at least 80 to produce a catalytic product stream comprising monomers. An embodiment of the disclosure is one, any or all of prior embodiments in this paragraph up through the first embodiment in this paragraph further comprising heating the plastic feed stream to a temperature of at least 500° C. An embodiment of the disclosure is one, any or all of prior embodiments in this paragraph up through the first embodiment in this paragraph wherein the catalyst is a molecular sieve. An embodiment of the disclosure is one, any or all of prior embodiments in this paragraph up through the first embodiment in this paragraph wherein the zeolitic catalyst has an MFI structure. An embodiment of the disclosure is one, any or all of prior embodiments in this paragraph up through the first embodiment in this paragraph wherein the silica-to-alumina ratio is at least 200. An embodiment of the disclosure is one, any or all of prior embodiments in this paragraph up through the first embodiment in this paragraph further comprising producing at least 75 wt % gas. An embodiment of the disclosure is one, any or all of prior embodiments in this paragraph up through the first embodiment in this paragraph further comprising contacting the plastic feed with a diluent gas at high temperature to provide the vaporized pyrolysis stream and contacting the vaporized pyrolysis stream in diluent gas with the catalyst. An embodiment of the disclosure is one, any or all of prior embodiments in this paragraph up through the first embodiment in this paragraph wherein the feed is a polyolefin. An embodiment of the disclosure is one, any or all of prior embodiments in this paragraph up through the first embodiment in this paragraph further comprising quenching the catalytic product stream to below 450° C.

A second embodiment of the disclosure is a process for converting plastics to monomers comprising heating a plastic feed to a temperature of greater than 600° C. to pyrolyze the plastic feed to provide a vaporized pyrolysis stream; contacting the vaporized plastic pyrolysis stream with a catalyst having a silica-to-alumina ratio of more than 50 to produce a catalytic product stream comprising monomers. An embodiment of the disclosure is one, any or all of prior embodiments in this paragraph up through the second embodiment in this paragraph further comprising heating the plastic feed stream to a temperature of at least 630° C. An embodiment of the disclosure is one, any or all of prior embodiments in this paragraph up through the second embodiment in this paragraph wherein the catalyst has an MFI structure. An embodiment of the disclosure is one, any or all of prior embodiments in this paragraph up through the second embodiment in this paragraph wherein the silica-to-alumina ratio is at least 300. An embodiment of the disclosure is one, any or all of prior embodiments in this paragraph up through the second embodiment in this paragraph further comprising producing at least 75 wt % gas. An embodiment of the disclosure is one, any or all of prior embodiments in this paragraph up through the second embodiment in this paragraph further comprising contacting the plastic feed with a gas at high temperature to provide the vaporized pyrolysis stream and contacting the vaporized pyrolysis stream in diluent gas with the catalyst. An embodiment of the disclosure is one, any or all of prior embodiments in this paragraph up through the second embodiment in this paragraph further comprising quenching the catalytic product stream to below 450° C.

A third embodiment of the disclosure is a process for converting plastics to monomers comprising contacting a plastic feed with a catalyst having a silica-to-alumina ratio of at least 40 at reaction temperature of at least 500° C. to produce a catalytic product stream comprising monomers. An embodiment of the disclosure is one, any or all of prior embodiments in this paragraph up through the third embodiment in this paragraph further comprising a reaction temperature of at least 530° C. An embodiment of the disclosure is one, any or all of prior embodiments in this paragraph up through the third embodiment in this paragraph wherein the catalyst has an MFI structure. An embodiment of the disclosure is one, any or all of prior embodiments in this paragraph up through the third embodiment in this paragraph further comprising quenching the catalytic product stream to below 450° C.

Without further elaboration, it is believed that using the preceding description that one skilled in the art can utilize the present disclosure to its fullest extent and easily ascertain the essential characteristics of this disclosure, without departing from the spirit and scope thereof, to make various changes and modifications of the disclosure and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

1. A process for converting plastics to monomers comprising:
heating a plastic feed to a temperature of about 450 to about 700° C. to pyrolyze the plastic feed to provide a vaporized pyrolysis stream;
contacting the vaporized plastic pyrolysis stream with a catalyst having a silica-to-alumina ratio of at least 80 to produce a catalytic product stream comprising monomers.

2. The process of claim 1 further comprising heating the plastic feed stream to a temperature of at least 500° C.

3. The process of claim 1 wherein said catalyst is a molecular sieve.

4. The process of claim 1 wherein said zeolitic catalyst has an MFI structure.

5. The process of claim 5 wherein said silica-to-alumina ratio is at least 200.

6. The process of claim 1 further comprising producing at least 75 wt % gas.

7. The process of claim 1 further comprising contacting said plastic feed with a diluent gas at high temperature to provide said vaporized pyrolysis stream and contacting said vaporized pyrolysis stream in diluent gas with said catalyst.

8. The process of claim 1 wherein said feed is a polyolefin.

9. The process of claim 1 further comprising quenching said catalytic product stream to below 450° C.

10. A process for converting plastics to monomers comprising:
heating a plastic feed to a temperature of greater than 600° C. to pyrolyze the plastic feed to provide a vaporized pyrolysis stream;
contacting the vaporized plastic pyrolysis stream with a catalyst having a silica-to-alumina ratio of more than 50 to produce a catalytic product stream comprising monomers.

11. The process of claim 10 further comprising heating the plastic feed stream to a temperature of at least 630° C.

12. The process of claim 10 wherein said catalyst has an MFI structure.

13. The process of claim 10 wherein said silica-to-alumina ratio is at least 300.

14. The process of claim 10 further comprising producing at least 75 wt % gas.

15. The process of claim 10 further comprising contacting said plastic feed with a gas at high temperature to provide said vaporized pyrolysis stream and contacting said vaporized pyrolysis stream in diluent gas with said catalyst.

16. The process of claim 1 further comprising quenching said catalytic product stream to below 450° C.

17. A process for converting plastics to monomers comprising:
contacting a plastic feed with a catalyst having a silica-to-alumina ratio of at least 40 at reaction temperature of at least 500° C. to produce a catalytic product stream comprising monomers.

18. The process of claim 17 further comprising a reaction temperature of at least 530° C.

19. The process of claim 17 wherein said catalyst has an MFI structure.

20. The process of claim 1 further comprising quenching said catalytic product stream to below 450° C.
